(19) United States
(12) Patent Application Publication
(10) Pub. No.: US 2022/0361697 A1
(43) Pub. Date: Nov. 17, 2022

(54) STRAW STRUCTURE

(71) Applicant: TIMAS TITAN CO., LTD., Taipei City (TW)

(72) Inventors: Han-Chun Hsieh, Taipei City (TW); Han-Chieh Hsieh, Taipei City (TW)

(21) Appl. No.: 17/539,102

(22) Filed: Nov. 30, 2021

(30) Foreign Application Priority Data
May 12, 2021 (TW) 110205326

(51) Int. Cl. A47G 21/18

(52) U.S. Cl. CPC A47G 21/18

(57) ABSTRACT

A straw structure is provided. The straw structure includes a straw body, and the straw body has a first beveled edge at an opening of a bottom end of the straw body and a second beveled edge connected to a rear side of the first beveled edge. A puncturing end opening is formed at the first beveled edge and a side edge of the straw body, and the bottom end of the straw body has a beveled opening formed at the second beveled edge. The puncturing end opening is an acute angle that is less than 90 degrees, and a slope of the second beveled edge is greater than a slope of the first beveled edge.

### CROSS-REFERENCE TO RELATED PATENT APPLICATION

This application claims the benefit of priority to Taiwan Patent Application No. 110205326, filed on May 12, 2021. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a straw structure, and more particularly to a modified straw structure for a user to suck a drink from a cup.

### BACKGROUND OF THE DISCLOSURE

With the recent pursuit of environmental protection and attempts at decreasing the use of disposable plastic containers, use of thermos bottles or tumblers for carrying water or beverages has become a trend in the modern society. In addition, in order to reduce the use of the plastic straws, people have turned to using reusable straws made of metal or glass to drink water or beverages.

However, since a conventional non-disposable straw usually has a bottom portion that has an angle of 90 degrees (more specifically, a corner of the bottom portion is usually 90 degrees,) the bottom portion of the conventional straw cannot easily puncture a sealing film of a drink cup, such that the use of the conventional straw is inconvenient. In order to overcome the above issue, the conventional straw may have a sharp end portion at the bottom portion thereof. However, since an angle formed by the sharp end portion is usually very small (i.e., an acute angle), and the conventional non-disposable straw is usually made of a hard material such as glass or metal, a puncturing end of the conventional straw is often as sharp as a needle or a knife and can easily puncture into the user's body or an accommodating pouch for carrying the conventional straw. Therefore, the conventional straw is inconvenient to use.

Based on the above reasons, the conventional straw has many inconveniences in use, and how to modify the structure of the conventional straw to overcome said inconveniences has become an important issue in the related art.

### SUMMARY OF THE DISCLOSURE

In response to the above-referenced technical inadequacies, the present disclosure provides a modified straw structure to improve upon the inconveniences of the conventional straw.

In one aspect, the present disclosure provides a straw structure including a straw body. The straw body has a cylindrical rod shape. The straw body defines a central axis and a horizontal axis perpendicular to the central axis, two ends of the straw body at the central axis are respectively defined as an upper end and a bottom end, and the straw body has a hollow passage penetrating a center of the straw body from the upper end to the bottom end. The bottom end of the straw body has a first beveled edge located at a side edge of the straw body at an opening of the bottom end and a second beveled edge connected to a rear side of the first beveled edge. An angle between the first beveled edge and the horizontal axis is defined as a first beveled angle, and an angle between the second beveled edge and the horizontal axis is defined a second beveled angle. The bottom end of the straw body has a puncturing end opening at a position where the first beveled edge and the side edge of the straw body connect to each other, the bottom end of the straw body has a beveled opening at the second beveled edge, an angle of the puncturing end opening between the first beveled edge and the side edge of the straw body is less than 90 degrees, and the first beveled angle is less than the second beveled angle.

Therefore, the bottom portion of the straw body of the present disclosure can be used to easily puncture a sealing film of a cup body. The puncturing end can be prevented from easily stabbing into a user or an accommodating pouch thereof, and the straw structure can be easily carried.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic perspective view of a straw structure according to the present disclosure;

FIG. 2 is a schematic sectional view of the straw structure according to the present disclosure;

FIG. 3 is a schematic enlarged sectional view of a bottom portion of the straw structure according to the present disclosure;

FIG. 4 is schematic planar view from a rear side of the straw structure according to the present disclosure; and

FIG. 5 and FIG. 6 are schematic views of the straw structure in use according to the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

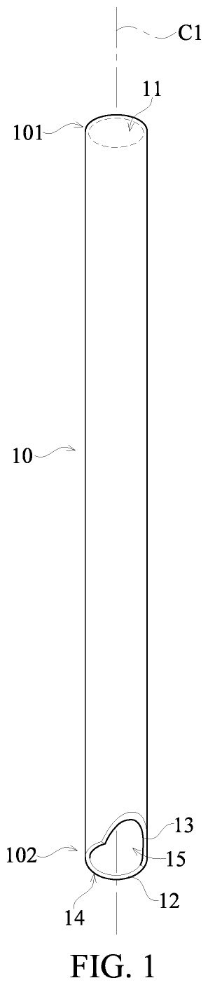

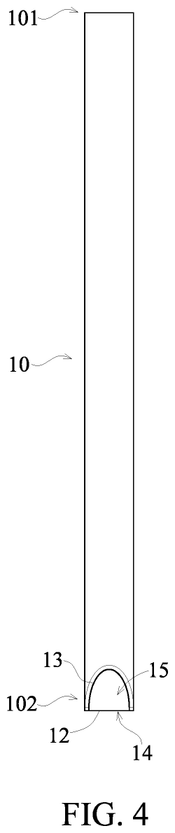

Referring to FIG. 1 to FIG. 6, an embodiment of the present disclosure provides a straw structure, the straw structure includes a straw body 10, the straw body 10 is a hollow tube in a cylindrical rod shape, and the straw body 10 can define a central axis C1 passing through a center of the straw body 10. Two ends of the straw body 10 at the central axis C1 are respectively defined as an upper end 101 and a bottom end 102, and the straw body 10 has a hollow passage 11 penetrating the center of the straw body 10 from the upper end 101 to the bottom end 102.

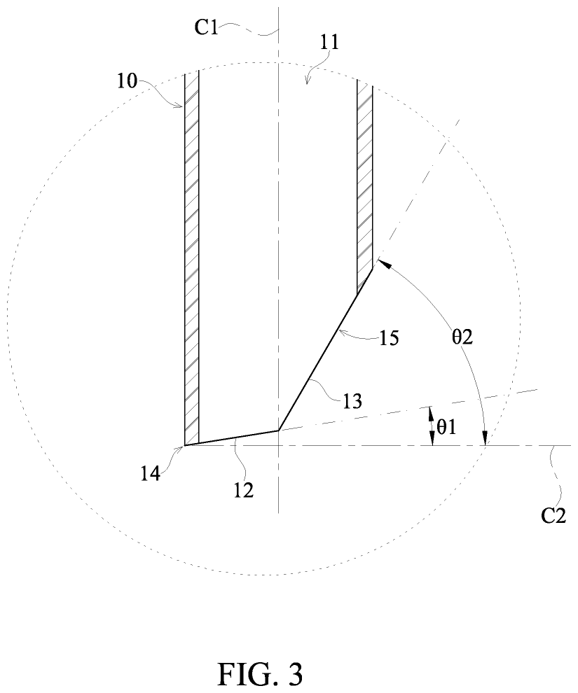

The bottom end 102 of the straw body 10 has a first beveled edge 12 located at a side edge of the straw body 10 at an opening of the bottom end 102 and a second beveled edge 13 connected to a rear side of the first beveled edge 12. As shown in FIG. 3, an angle between the first beveled edge 12 and a horizontal axis C2 perpendicular to the central axis C1 is defined as a first beveled angle θ1, and an angle between the second beveled edge 13 and the horizontal axis C2 is defined a second beveled angle θ2. A puncturing end opening is formed at a position where the first beveled edge 12 and the side edge of the straw body 10 at the bottom end 102 connect to each other, and the bottom end 102 of the straw body 10 has a beveled opening 15 at the second beveled edge 13.

A diameter of the straw body 10 is within a range from 6 millimeters to 15 millimeters, so that chewables having a large diameter can pass through the hollow passage 11 of the straw body 10. Therefore, the straw structure of the present disclosure can be suitable for being used to sip drinks containing chewables such as tapioca balls, konjac, or fruit pieces.

In addition, an angle between the first beveled edge 12 and the horizontal axis C2 is a tilt angle within a range from 5 degrees to 30 degrees, the second beveled angle θ2 is a tilt angle within a range from 30 degrees to 60 degrees. Through the above configuration, an angle of the puncturing end opening 14 is within a range from 60 degrees to 85 degrees. Accordingly, the angle of the puncturing end opening 14 is an acute angle less than 90 degrees, and the angle of the puncturing end opening 14 is able to puncture a sealing film of a drink cup while not being so sharp as to easily puncture the skin of a user. In addition, a position of the bottom end of the straw body 10 at the beveled opening 15 has a greater tilt angle, and when the straw body 10 is inserted into a drink container body 20, a gap between the second beveled edge 13 and a bottom portion of the drink container body 20 is great enough for chewables (e.g., tapioca balls, konjac, or fruit pieces) to be easily sucked into the straw body 10 from the beveled opening 15.

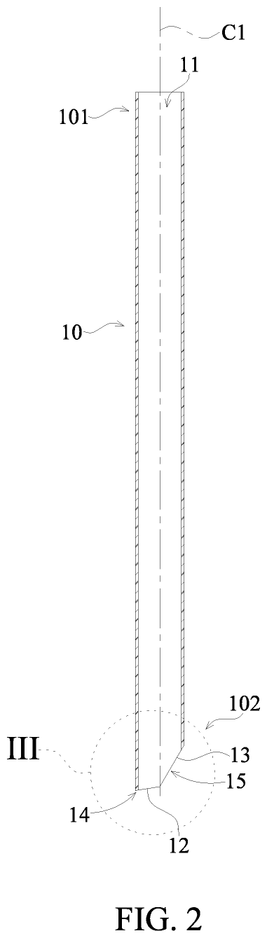

In addition, as shown in FIG. 2 and FIG. 3, in the present embodiment, the side edge of the straw body 10 near the puncturing end opening 14 is defined as a first side edge, an intersection where the first beveled edge 12 and the second beveled edge 13 intersect is located at a position of the bottom end 102 of the straw body 10 between the first side edge and the central axis C1 of the straw body 10. Through the above configuration, it can be ensured that a width of the second beveled edge 13 is equal to or greater than a width of the first beveled edge 12, and accordingly, a width of the beveled opening 15 can be large enough.

The straw structure of the present disclosure is a non-disposable straw that can be reused. Therefore, the straw body 10 is one of a plastic straw body, a glass straw body, and a metal straw body. Accordingly, the straw body 10 has a great enough structural strength for being reused and is not easily damaged. In a preferable embodiment of the present disclosure, the straw body 10 is a tube made of food grade or medical grade metal materials (e.g., stainless steel, or titanium), and in a further embodiment of the present disclosure, the straw body 10 is a titanium tube.

In addition, in an embodiment of the present disclosure, a surface of the straw body 10 can have an antibacterial and anti-sticking film. Preferably, the antibacterial and anti-sticking film is a titanium-based oxide layer or a titanium-based nitride (i.e., TiO₂ or TiN) layer. Therefore, the straw body 10 can be easily cleaned, and bacterial contamination can be prevented.

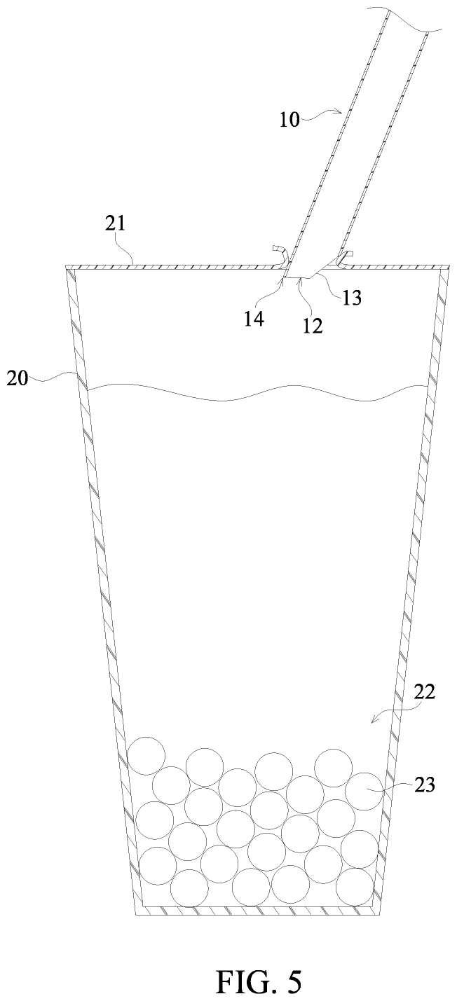

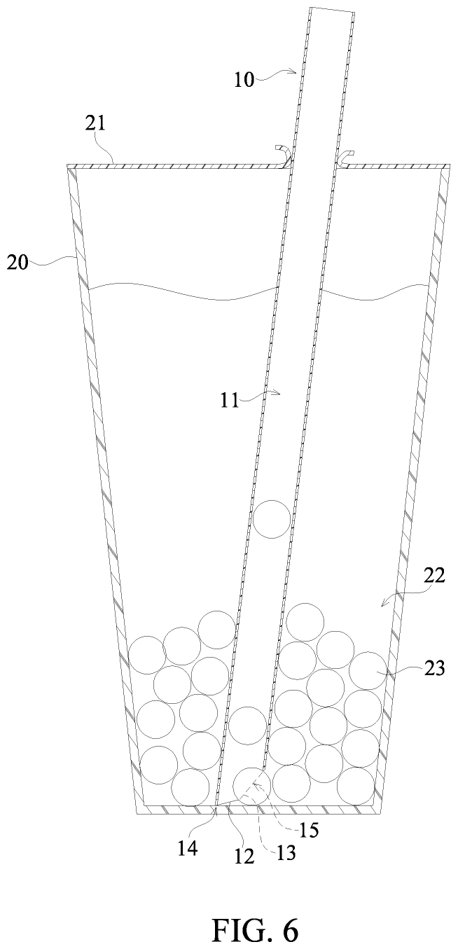

Referring to FIG. 5 and FIG. 6, FIG. 5 and FIG. 6 show a method for using the straw structure of the present disclosure. As shown in FIG. 5, through the first beveled edge 12 located at the bottom end 102 of the straw body 10 of the present disclosure, the puncturing end opening 14 that is in an acute angle forms at a side edge of a bottom-most position of the straw body 10. Therefore, the user can easily use the puncturing end opening 14 to puncture the sealing film 21 of the drink container body 20, and the straw body 10 can be easily inserted into the drink container body 20. In addition, when the straw body 10 is inserted into the bottom-most position of the drink container body 20, an angle is formed between the second beveled edge 13 and a bottom surface of the drink container body 20, so that the beveled opening 15 of the straw body 10 at the bottom end 102 faces toward a side of the straw body 10, and a diameter of the beveled opening 15 is great enough so that the beveled opening 15 is able to accommodate chewables (i.e., tapioca balls, konjac, or fruit pieces) having a large diameter. Therefore, the chewables 23 in the drink 22 can be easily sucked into the hollow passage 11 through the beveled opening 15.

[Beneficial Effects of the Embodiment]

In conclusion, through the puncturing end opening and the beveled opening formed at the first beveled edge and the second beveled edge located at the bottom portion of the straw body, the straw structure of the present disclosure can easily puncture the sealing film of the drink container body and can provide easy passage for chewables such as tapioca balls, konjac, or fruit pieces. Therefore, the straw structure of the present disclosure is suitable for being used to sip beverages such as bubble tea.

Further, since the straw body of the present disclosure has the first beveled edge and the second beveled edge having two different slopes at the bottom portion, the puncturing end opening at the bottom portion of the straw body can be used to easily puncture the sealing film of the drink container body, and the puncturing end opening can be prevented from easily stabbing into a user or an accommodating pouch thereof. Therefore, the straw structure of the present disclosure can be easily used and can be safely carried.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A straw structure, comprising:
a straw body having a cylindrical rod shape, wherein the straw body defines a central axis and a horizontal axis perpendicular to the central axis, two ends of the straw body at the central axis are respectively defined as an upper end and a bottom end, and the straw body has a hollow passage penetrating a center of the straw body from the upper end to the bottom end;
wherein the bottom end of the straw body has a first beveled edge located at a side edge of the straw body at an opening of the bottom end and a second beveled edge connected to a rear side of the first beveled edge, and wherein an angle between the first beveled edge and the horizontal axis is defined as a first beveled angle, and an angle between the second beveled edge and the horizontal axis is defined a second beveled angle, and
wherein the bottom end of the straw body has a puncturing end opening at a position where the first beveled edge and the side edge of the straw body connect to each other, the bottom end of the straw body has a beveled opening at the second beveled edge, an angle of the puncturing end opening between the first beveled edge and the side edge of the straw body is less than 90 degrees, and the first beveled angle is less than the second beveled angle.

2. The straw structure according to claim 1, wherein the first beveled angle is within a range from 5 degrees to 30 degrees, and the second beveled angle is within a range from 30 degrees to 60 degrees.

3. The straw structure according to claim 1, wherein a diameter of the straw body is within a range from 6 millimeters to 15 millimeters.

4. The straw structure according to claim 1, wherein the side edge of the straw body near the puncturing end opening is defined as a first side edge, an intersection where the first beveled edge and the second beveled edge intersect is located at a position of the bottom end of the straw body between the first side edge and the central axis of the straw body.

5. The straw structure according to claim 1, wherein the straw body is one of a plastic straw body, a glass straw body, and a metal straw body.

6. The straw structure according to claim 5, wherein the straw body is a titanium straw body.

7. The straw structure according to claim 5, wherein a surface of the straw body has an antibacterial and anti-sticking film.

8. The straw structure according to claim 7, wherein the antibacterial and anti-sticking film is a titanium-based oxide layer or a titanium-based nitride layer.
